(19) United States
(12) Patent Application Publication
(10) Pub. No.: US 2004/0096461 A1
(43) Pub. Date: May 20, 2004

(54) Chimeric multivalent polysaccharide conjugate vaccines

(75) Inventors: Francis Michon, Bethesda, MD (US); John Kim, Arbutus, MD (US); Arun Sarkar, Olney, MD (US); Catherine Uitz, Arlington, VA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Baxter Healthcare Corporation, Irvine, CA

(21) Appl. No.: 10/630,223

(22) Filed: Jul. 30, 2003

Related U.S. Application Data
(60) Provisional application No. 60/399,949, filed on Jul. 30, 2002.

(51) Int. Cl. A61K 39/116; C07K 14/195

(52) U.S. Cl. 424/203.1; 530/395

(57) ABSTRACT

The present invention provides multivalent chimeric conjugate vaccine molecule and methods of using the conjugate to immunize subjects against bacterial infections. A conjugate molecule of the invention comprises multiple bacterial capsular polysaccharides linked to a carrier protein. Accordingly, the conjugate molecule provides immune protection against multiple types of bacteria in a single vaccines. In particular, conjugate molecules of the invention are used to prevent or attenuate Group B Streptococcus and meningococcal infections.

### CROSS-REFERENCES TO RELATED APPLICATIONS

This application claims benefit of priority to U.S. provisional application No. 60/399,949, filed Jul. 30, 2002, which application is herein incorporated by reference.

### BACKGROUND OF THE INVENTION

The present invention provides a multivalent conjugate molecule and methods of using the conjugate to immunize subjects against bacterial infections. A conjugate molecule of the invention comprises multiple bacterial capsular polysaccharides linked to a carrier protein. Accordingly, the conjugate molecule provides immune protection against multiple types of a particular bacteria in a single vaccine. A vaccine comprising such conjugate molecules also provides a protective immunogenic response that is equivalent to that obtained from a multivalent vaccine that is a mixture of single polysaccharides conjugated to carrier protein. In particular, conjugate molecules of the invention are used to prevent or attenuate Group B Streptococcus and Meningococcal infections.

A trivalent vaccine was previously described in U.S. Pat. No. 4,711,779. This vaccine included at least two bacterial capsular oligosaccharidic haptens from a gram-negative bacterium and a gram positive bacterium covalently bonded to a carrier protein, thereby producing a trivalent glycoproteinic molecule. Although the patent discloses that antibodies are produced in response to this vaccine in rabbits and that the rabbit antisera shows bactericidal activity on living strains of Neisseria meningitidis, there is no disclosure that such a vaccine elicits a protective immune response.

Recently, another conjugate molecule has been described in which carbohydrate antigens are combined in the same molecule (Allen et al., J. Am. Chem. Soc. 123:1890-1897, 2001). In this conjugate molecule, carbohydrate-based antigen domains are linked to pure amino acids. Amino acid coupling reactions are then used to link the domains together. In particular, the authors described a conjugate that includes three cancer-cell antigen carbohydrates linked via amino acids. This study also fails to disclose that the vaccine is effective in eliciting a protective immune response.

The present invention provides vaccines comprising multiple bacterial polysaccharides linked to a single carrier protein. As described herein, these vaccines elicit a protective immune response. Moreover, the degree of protection is equivalent to that obtained using a multivalent vaccine mixture of single polysaccharides linked to a carrier molecule. Thus, the present invention provides a vaccine that is not only equivalent in its efficacy to current multivalent vaccine mixtures, but is also more cost-effectively produced.

### BRIEF SUMMARY OF THE INVENTION

The present invention provides a multivalent conjugate vaccine comprising a carrier protein with at least three different bacterial capsular polysaccharides covalently linked to the carrier protein. The immunogenic molecule often comprises four, five, or six different bacterial capsular polysaccharides covalently linked to the carrier protein.

The carrier protein is typically selected from the group consisting of Cα, Cβ, tetanus toxoid, diphtheria toxoid, diphtheria toxoid analog CRM197, and a porin protein. In one embodiment, the bacterial capsular polysaccharides are different Group B Streptococcus capsular polysaccharides selected from the group consisting of type Ia, type Ib, type II, type III, type V, and type VIII. Frequently, the Group B Streptococcus capsular polysaccharides are type Ia, type III and type V and the carrier protein is Cβ.

In another embodiment, the bacterial capsular polysaccharides are Neisseria meningitidis capsular polysaccharides selected from the group consisting of A, B, C, W, and Y. Often, the Neisseria meningitidis capsular polysaccharides are B, C, and Y, or C, Y, and W-135; and the carrier protein is a tetanus toxoid or a porin, e.g., recombinant porin B.

In further embodiments, the immunogenic molecule includes bacterial capsular polysaccharides that are of a size of between 80 and 120 kilodaltons. In particular embodiments, between about 5 and 20% of the sialic acid residues of the bacterial capsular polysaccharides can be covalently linked to the carrier protein. Often, the bacterial capsular polysaccharides are present in equimolar amounts.

The invention also provides a method of preparing a multivalent immunogenic molecule, the method comprising covalently linking at least three different bacterial capsular polysaccharides to a carrier protein. In one embodiment, covalently linking the bacterial capsular polysaccharides to the carrier protein comprises steps of: (a) oxidizing the polysaccharides; and (b) coupling the oxidized polysaccharides to the carrier protein.

The polysaccharides can be coupled to the carrier protein by reductive animation. In an alternative embodiment, the polysaccharides are coupled to the carrier protein by a bispacer coupling with a linker.

In particular embodiments, the invention provides methods of preparing a conjugate molecule that comprises bacterial capsular polysaccharides that are different Group B Streptococcus capsular polysaccharides selected from the group consisting of type Ia, type Ib, type II, type III, type V, and type V. Often, the Group B Streptococcus capsular polysaccharides are type Ia, type III, and type V.

In some embodiments, about 5 and 20% of the sialic acid residues of the bacterial capsular polysaccharides are oxidized and about 5 and 20% of the sialic acid residues of the bacterial capsular polysaccharides are coupled to protein.

In additional embodiments, the methods of the invention are used to prepare a conjugate molecule wherein the bacterial capsular polysaccharides are Neisseria meningitidis capsular polysaccharide selected from the group consisting of A, B, C, W-135, and Y. Often the polysaccharides are B, C, and Y, or C, Y, and W-135; and the carrier protein is a tetanus toxoid or porin, e.g., recombinant porin B.

In another aspect, the invention provides a method of preventing or attenuating an infection in a mammal, the method comprising administering to the mammal a multivalent immunogenic molecule comprising a carrier protein with at least three different bacterial capsular polysaccharides covalently linked to the carrier protein, wherein the multivalent immunogenic molecule is administered in an amount sufficient to elicit protective antibodies against the bacterial capsular polysaccharides.

Often the multivalent immunogenic molecule is administered to prevent or attenuate an infection caused by Group B Streptococcus and the bacterial capsular polysaccharides of the immunogenic molecule are different Group B Streptococcus capsular polysaccharides selected from the group consisting of type Ia, type Ib, type II, type III, type V, and type VIII. The carrier protein is typically selected from the group consisting of Cα, Cβ, tetanus toxoid, and diphtheria toxoid. In particular embodiments the polysaccharides are type Ia, type III, and type V and the carrier protein is Cβ.

In another embodiments, the multivalent immunogenic molecule is administered to prevent or attenuate an infection caused by Neisseria meningitidis and the bacterial capsular polysaccharides of the immunogenic molecule are different Neisseria meningitidis capsular polysaccharides selected from the group consisting of A, B, C, W-135, and Y. Often, the Neisseria meningitidis capsular polysaccharides are B, C, and Y, or C, Y, and W-135; and the carrier protein is a tetanus toxoid or a porin such as recombinant porin B.

The invention also provides a method of preventing or attenuating an infection caused by a Group B Streptococcus in a mammal, the method comprising administering a multivalent immunogenic molecule comprising a carrier protein with at least three different bacterial capsular polysaccharides covalently linked to the carrier protein, wherein the bacterial capsular polysaccharides are different Group B Streptococcus capsular polysaccharides selected from the group consisting of type Ia, type Ib, type II, type III, type V, and type VIII; and, wherein the immunogenic molecule is administered to a pregnant female in an amount sufficient to confer immunity to the infection in utero to an offspring of the female. Often, the carrier protein is selected from the group consisting of Cα, Cβ, tetanus toxoid, and diphtheria toxoid. In a particular embodiment, the Group B Streptococcus capsular polysaccharides are type Ia, type III and type V and the carrier protein is Cβ.

The invention also provides a pharmaceutical composition comprising a multivalent immunogenic molecule comprising a carrier protein with at least three different bacterial capsular polysaccharides covalently linked to the carrier protein and a pharmacological acceptable carrier, wherein the multivalent immunogenic molecule is in an amount sufficient to elicit protective antibodies against the three different bacterial capsular polysaccharides. The carrier protein is frequently selected from the group consisting of Cα, Cβ, tetanus toxoid, and diphtheria toxoid. Are there other carrier proteins.

In a particular embodiment the bacterial capsular polysaccharides are different Group B Streptococcus capsular polysaccharides selected from the group consisting of type Ia, type Ib, type II, type III, type V, and type VIII. Often, the Group B Streptococcus capsular polysaccharides are type Ia, type III and type V.

### BRIEF DESCRIPTION OF THE DRAWINGS

FIG. 1 provides a schematic showing the preparation of a Group B Streptococcus chimeric conjugate vaccine.

FIG. 2 shows the structures of the repeating units of the Group B Streptococcus polysaccharides Ia, Ib, II, III and V.

FIG. 3 shows Molar Mass determinations by SEC-MALLS for GBS polysaccharides prior to being conjugated.

FIG. 4 shows the structure of an oxidized GBS polysaccharide having an aldehyde group in its terminal sialic acid.

FIG. 5 provides a schematic showing a conjugation reaction carried out by reductive amination.

FIG. 6 provides a table showing all expected methylated monosaccharides from methylation analysis in the types Ia, Ib, II, III, and V capsular polysaccharides.

FIG. 7 shows a chromatographic trace (GC) of PMAA (partially methylated alditol acetates) derivatives from a GBS mulitvalent chimeric (Ia, III, an V) conjugate.

FIG. 8 shows results of an ELISA competition experiment in vitro demonstrating that the type Ia polysaccharide in the chimeric conjugate competes for binding with a type Ia polysaccharide conjugate monovalent counterpart.

FIG. 9 shows results of an ELISA competition experiment in vitro demonstrating that the type III polysaccharide in the chimeric conjugate competes for binding with a type III polysaccharide conjugate monovalent counterpart.

FIG. 10 shows results of an ELISA competition experiment in vitro demonstrating that the type V polysaccharide in the chimeric conjugate competes for binding with a type V polysaccharide conjugate monovalent counterpart.

FIG. 11 shows that a chimeric Ia/III/V GBS vaccine conjugate elicits a protective immune response in the neonatal mouse model similar to that of a combination vaccine composed of a mixture of the individual serotype Ia/III/V polysaccharide conjugates.

FIG. 12 shows a chromatogram (GC) of trimethylsilyl methyl glycoside derivatives obtained from a meningococcal C/Y/W-135 chimeric conjugate.

FIG. 13 shows that a meningococcal chimeric vaccine conjugate elicits a protective immune response similar to that elicited by a combination vaccine composed of a mixture of the individual serogroup CWY polysaccharide conjugates.

### DETAILED DESCRIPTION OF THE INVENTION

Definitions

A “bacterial capsular polysaccharide” is a polysaccharide that is the predominant carbohydrate present in a capsule of a bacteria. The term includes functional derivatives or variants of the polysaccharides. For example, a Group B Streptococcus polysaccharide is any group B-specific or type-specific polysaccharide.

The term “carrier”, “carrier protein”, or “carrier polypeptide” are used interchangeably to refer to a polypeptide moiety to which the polysaccharide antigens are covalently linked. A carrier protein is often immunogenic and therefore also contributes to the “valency” of the vaccine. Linkage to the carrier protein typically increases the antigenicity of the conjugated carbohydrate molecules. The carrier protein may be from the same target organism as the polysaccharides linked to it or may be from a different organism.

The terms “polypeptide”, “oligopeptide”, “peptide”, and “protein” are used interchangeably herein to refer to a polymer of amino acid residues. The terms apply to amino acid polymers in which one of more amino acid residue is an artificial chemical analog of a corresponding naturally occurring amino acid as well as to naturally occurring amino acid polymers. The term also includes variants on the traditional peptide linkage joining the amino acids making up the polypeptide.

“Conservatively modified variants”, “analogs”, or “functional derivative” refer to an amino acid sequence that includes a modification to the sequence compared to the native or naturally sequence, but retains the same biological function, i.e., the ability to act as a carrier protein that is at least equal to that of the native molecule. One of skill recognizes that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. For example, the following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7)Serine (S), Threonine (T); and 8) Cysteine (C), Methionine (M) (see, e.g., Creighton, Proteins (1984)).

A “multivalent” molecule or vaccine comprises more than one antigenic epitope. For example, multivalent vaccines of the invention often comprise at least three different bacterial polysaccharides conjugated to a single carrier protein. Such a vaccine therefore comprises four antigenic determinants and is a tetravalent vaccine.

The term “chimeric” as used herein refers to a multivalent vaccine in which at least two different polysaccharides are conjugated to the carrier.

“Linked” “joined” or “conjugated” refer to covalent linkage of a carbohydrate to the carrier protein. The covalent linkage can be direct or indirect, e.g., linked through a spacer molecule.

The term “purified” means substantially free of the various protein, lipid, and carbohydrate components that naturally occur with the polysaccharide. In particular, purified oligosaccharide, or bacterial capsule polysaccharide, is substantially free of intact polysaccharide capsule, or fragments of it having a molecular weight above 100,000. Traces of foreign components that may remain in the purified polysaccharide do not interfere with the use of the purified material in a vaccine or as an antigen. The term “purified” does not exclude synthetic oligosaccharide preparations retaining artifacts of their synthesis; nor does the term exclude preparations that include some impurities, so long as the preparation exhibits reproducible polysaccharide characterization data, for example molecular weight, sugar residue content, sugar linkages, chromatographic response, and immunogenic behavior.

The term “pharmacologically acceptable” or “pharmaceutically acceptable” refers to a composition that is tolerated by a recipient patient.

A “pharmaceutical excipient” is administered as a component of a vaccine in conjunction with the immunogenic multivalent molecule. Excipients comprise a material such as an adjuvant, a carrier, pH-adjusting and buffering agents, tonicity adjusting agents, wetting agents, preservative, and the like.

A “protective immune response” or “therapeutic immune response” refers to a B lymphocyte and/or T lymphocyte response to a conjugate molecule of the invention that prevents or at least partially arrests or attenuates a bacterial infection and/or disease symptoms or progression caused by the infection. The immune response can include an antibody response that has been facilitated by the stimulation of helper T cells.

A “patient” or “recipient” is an animal that is a target of vaccination with a conjugate molecule of the invention. The patient is most often a human.

Introduction

Vaccines to immunize against bacterial polysaccharides are well known in the art. These vaccines comprise purified bacterial capsular polysaccharides that are typically linked to a carrier. Such vaccines for Group B Streptococcus are disclosed, e.g., in U.S. Pat. Nos. 5,993,825; 5,968,521; 5,908,629; 5,858,362; 5,847,081; 5,843,461; 5,843,444; 5,8200,850; and 5,705,580). Similar vaccines have also been developed for Neisseria meningitidis (see, e.g. U.S. Pat. Nos. 5,597,572; 5,425,946; 5,811,102, and 6,013,267). Polysaccharide vaccines are typically linked to a protein carrier in order to provide optimized immunogenicity.

The present invention provides multivalent vaccine conjugate molecules that include multiple bacterial capsular polysaccharides linked to a single carrier protein. The invention also provides methods of producing such vaccines and methods of using the vaccines to obtain protective immunization. Multivalent vaccines that are mixtures of single polysaccharides conjugated to a carrier molecule are well-known in the art and used to confer immune protection against multiple bacterial types. The present invention provides a multivalent vaccine conjugate molecule that is as effective as a multivalent vaccine mixture in eliciting a protective immune response.

In particular, the invention provides vaccines and methods of using the vaccines to provide protective immunity against Group B Streptococcus and Neisseria meningitidis.

Bacterial Capsular Polysaccharides

Bacterial capsular polysaccharides are the carbohydrate moieties that comprise the capsule coating bacteria. These have been extensively evaluated for many different bacteria. The vaccines of the invention comprise purified polysaccharides or polysaccharide derivatives that are modified versions of the polysaccharide that typically exhibit increased immunogenicity relative to the unmodified version of the polysaccharide.

Many different bacterial capsular polysaccharides can be used in the methods of the invention. These include polysaccharides from bacteria including, but not limited to gram-positive bacteria such as Streptococci, Staphylococci, Enterococci, Bacillus, Corynebacterium, Listeria, Erysipelothrix, and Clostridium. Non-limiting examples of gram-negative bacteria for use with this invention include Haemophilus influenzae, Neisseria meningitidis and Escherichia coli. The polysaccharides are typically isolated from Group B Streptococcus types, as further described below; Neisseria meningitidis polysaccharides, further described below; Hemophilus influenzae polysaccharides, such as serotype b, Streptococcus pneumonia polysaccharides including types 6A, 6B, 10A, 11 A, 18C, 19A, 19f, 20, 22F, and 23F, and various Escherichia coli polysaccharides including K1, K2, K12, K13, K92, and K100 polysaccharides.

The polysaccharide capsule of Group B Streptococcus is well characterized and has been shown to play a role in both virulence and immunity (Kasper, et al., Infect. Dis. 153:407-415, 1986). Group B streptococci can be further classified into several different types based on the bacteria's capsular polysaccharide. Types Ia, Ib, II, III, IV, V, VI, VII, and VIII account for most of the pathogenicity due to group B infection, with group B streptococci types Ia, Ib, II, III, and V representing over 90% of all reported cases. The structure of each of these various type polysaccharides has been characterized (19-22, 44). The recognized Group B Streptococcus types and subtypes have chemically related but antigenically distinct capsular polysaccharides having a repeating structure composed of galactose, glucose, N-acetyl glucosamine, and N-acetyl-neuraminic (sialic) acid.

Neisseria meningitidis is a causative agent of bacterial meningitis and sepsis. Meningococci are divided into serological groups based on the immunological characteristics of capsular and cell wall antigens. Currently recognized serogroups include A, B, C, D, W-135, X, Y, Z and 29E. The polysaccharides responsible for the serogroup specificity have been purified from several of these groups, including A, B, C, D, W-135 and Y.

The polysaccharides that are incorporated into a conjugate multivalent molecule of the invention include polysaccharide derivatives, i.e., modified polysaccharides, as well as the native forms purified from the bacteria. Such modified polysaccharides often exhibit enhanced antigenicity relative to the native purified polysaccharide. Various modifications of bacterial capsular polysaccharides are well known in the art and include such modifications as N-propionylation and de-O-acetylation.

For example, the capsular polysaccharide type B from Neisseria meningitidis in its native form exhibits little antigenicity. Modified forms are therefore often used in vaccines to circumvent the poor immunogenicity of the native carbohydrate. Modifications of type B polysaccharide include C₃-C₈ N-acyl-substituted polysaccharide derivatives, which have been described e.g., in EP Publication No. 504,202 B, to Jennings et al. Similarly, U.S. Pat. No. 4,727,136 to Jennings et al. describes an N-propionylated polysaccharide type B in which N-propionyl groups are substituted for N-acetyl groups. The de-O-acetylation of group C meningococcal polysaccharides to enhance immunogenicity is described in U.S. Pat. No. 5,425,946. Methods for producing these derivatives are disclosed in the cited references.

Bacterial capsular polysaccharides can be purified in a variety of ways. Large-scale production of capsular polysaccharides and capsular polysaccharide conjugate vaccines, requires adequate supplies of purified capsular polysaccharides. Purification techniques that are particular useful in the invention yield polysaccharides that are uniform in size and reproducibly exhibit the same immunogenic properties. Methods for isolating capsular polysaccharides from bacterial cells include treatment of cells with the enzyme mutanolysin, which cleaves the bacterial cell wall to free the cell wall components. This procedure involves treating cell lysates with additional enzymes to remove proteins and nucleic acids and purification by differential precipitation and chromatography.

More efficient, higher yielding and simpler means of obtaining purified capsular polysaccharides are also available. For example, U.S. Pat. No. 6,248,570 describes a base-extraction method to obtain large quantities of capsular polysaccharides from cultures of bacteria. Following treatment with base, the polysaccharides are subjected to ultrafiltration to remove proteins and nucleic acids, thereby providing a polysaccharide preparation of relatively uniform molecular weight and free of contaminants. The polysaccharides can then be prepared for conjugation to the carrier protein, via a direct or indirect linkage, as further described below.

Carrier Proteins

Any number of carrier proteins can be used in the invention. The carrier, when introduced into the recipient animal, e.g., a human, typically increases the immunogenicity of the linked polysaccharides but may also elicit antibodies that are capable of reacting to a protein expressed by the bacteria from which is derived. Conjugation of the polysaccharides to the carrier usually converts the immune response to the polysaccharide, most often T-cell independent, to one that is T-cell dependent. The carrier protein can have the native amino acid sequence or can be a functional derivative or conservative modification of the native amino acid sequence. The term functional derivative includes fragments of a native protein, or variants of a native sequence, e.g., proteins that have changes in amino acid sequence, but retain the ability to elicit an immunogenic, virulence or antigenic property as exhibited by the native protein).

Various carrier proteins and analogs of the carrier proteins are well known in the art. These include, but are not limited to, carriers disclosed in U.S. Pat. No. 5,425,946, e.g., tetanus toxoid; non-toxic diphtheria toxoid and analogs, e.g., CRM197; the C protein of group B Streptococcus; and the outer membrane protein (porin protein) of Neisseria meningitidis. Suitable proteins can readily be identified by those of skill in the art.

One example of a carrier protein that is often used is a non-toxic diphtheria toxin analog, CRM197. The CRM197 protein is a nontoxic form of diphtheria toxin, which is produced by C. diphtheriae infected by the nontoxigenic phage β197ₜₒₓ-created by nitrosoguanidine mutagenesis of the toxigenic Corynephage β. (see, e.g., Uchida. et al., Nature New Biology 233:8-11, 1971). This carrier protein and other diphtheria toxin variants are widely used in the art and can be used for the preparation of many protein-polysaccharide conjugates (see, e.g, U.S. Pat. Nos. 4,761,283 and 5,614,382).

In further examples, such as a multivalent conjugate molecules that comprise Group B Streptococcus capsular polysaccharide, a Cα or Cβ carrier is often used. The C protein(s) are a group of a cell surface associated protein antigens of Group B Streptococcus (see, e.g., Wilkinson et al., J. Bacteriol. 97:629-634 (1969), Wilkinson, H. W, et al., Infec. and Immun. 4:596-604 (1971)). Two antigenically distinct populations of C proteins have been described, those that are sensitive to degradation by pepsin but not by trypsin, Cα and those that are sensitive to degradation by both pepsin and trypsin, Cβ. Method of producing Cα and Cβ and analogs of the proteins are described, e.g., in U.S. Pat. No. 5,908,629.

Porins may also be used as carriers. The meningococcal porins are divided into three major classifications, Class 1, 2, and 3 (Frasch et al., Rev. Infect. Dis. 7:504-510, 1985). Each meningococcus contains one of the alleles for either a Class 2 porin gene or a Class 3 porin gene but not both (see, e.g., Feavers et al., Infect. Immun. 60:3620-3629, 1992; and Murakani et al., Infect. Immun. 57:2318-2323, 1989). Methods of preparing porin proteins and analogs are known in the art. In particular, methods of expressing the outer membrane protein meningococcal group B porin proteins, por B, are described in U.S. Pat. Nos. 6,013,267 and 5,439,808 to Blake et al.

Conjugation of Polysaccharides to Carrier Proteins

Any method of covalently linkage may be employed to conjugate the purified polysaccharide components to the carrier, including both direct and indirect methods. Such methods are well known in the art (see, e.g., Jacob, et al., Eur. J. Immunol. 16:1057-1062, 1986; Parker et al., In: Modern Approaches to Vaccines, Chanock, et al., eds, pp. 133-138, Cold Spring Harbor Laboratory, Cold Spring Harbor, N.Y., 1983; Zurawski et al., J. Immunol. 121:122-139, 1978; Klipstein et al., Infect. Immun. 37:550-557, 1982; Bessler et al., Immunobiol. 170:239-244, 1985; Posnett et al., J. Biol. Chem. 263:1719-1725, 1988; Ghose et al., Molec. Immunol. 25:223-230, 1988; European Patent Publications 245,045 and 206,852; and U.S. Pat. Nos. 4,356,170, 4,673,574, 4761,283, 4,789,735, and 4,619,828).

Various techniques are known in the art to facilitate coupling of proteins and polysaccharides (see, e.g., Dick, et al., “Glyconjugates of Bacterial Carbohydrate Antigens: A Survey and Consideration of Design and Preparation Factors,” Conjugate Vaccines, Eds. Cruse, et al, Karger, Basel, 1989, beginning at page 48). As one example of a protein-polysaccharide coupling technique, the use of organic cyanylating reagents, such as 1-cyano-4-(dimethylamino)-pyridinium tetrafluoroboratehave been developed (see, e.g., U.S. Pat. No. 5,651,971

Often, the conjugates are produced by reductive amination, i.e., reacting the reducing end groups of the bacterial capsular polysaccharides to primary amino groups of the carrier protein by reductive amination. The reducing groups can be formed by selective hydrolysis or specific oxidative cleavage, or a combination of both. Often, the polysaccharide is conjugated to the carrier protein by the method of Jennings et al., U.S. Pat. No. 4,356,170, which involves controlled oxidation of the polysaccharide with periodate followed by reductive amination with the carrier protein. For example, a Group B Streptococcus capsular polysaccharide is purified, N-acetylated and subjected to periodate oxidation sufficient to introduce an aldehyde group into two or more terminal sialic acid residues linked to the backbone of the polysaccharide. The oxidized polysaccharide is conjugated to the carrier through reductive amination to generate a secondary amine bond between the capsular polysaccharide and the protein.

Often, in preparing the conjugate molecules for linkage to the protein carrier via reductive amination, equimolar amounts of the purified polysaccharides are mixed and oxidized to the extent that 5%-20% of the terminal sialic acid residues are oxidized. The mixture is then conjugated to the carrier, e.g., using NaBH₃CN and the conjugate molecule purified.

The conjugate vaccines of the invention are not limited to those produced via reductive amination or other methods of direct linkage of the polysaccharides to the protein moiety. Thus, the vaccines may also be produced by conjugating the polysaccharides indirectly to the carrier via any linking method known to those skill in the art such as spacer molecule. For example, an adipic dihydrazide spacer, as described by Schneerson, et al., J. Exp. Med., 1952:361-476, 1980, and in U.S. Pat. No. 4,644,059 can be employed to link the polysaccharide to the carrier. Other examples include the use of binary spacers as described by Marburg et al., J. Am. Chem. Soc., 108, 5282-5287, 1986, and in EP publication 0 467 714. The binary spacers are bigeneric spacers containing a thioether group and primary amine which form hydrolytically-labile covalent bonds with the polysaccharide and carrier protein.

Pharmaceutical Compositions and Administration of Vaccines

The conjugate molecules of the invention are typically administered as a pharmaceutical composition in a pharmacologically acceptable carrier. The compositions may comprise standard carriers, buffers or preservatives known to those in the art which are suitable for vaccines including, but not limited to, any suitable pharmaceutically acceptable carrier, such as physiological saline or other injectable liquids. Additives customary in vaccines may also be present, for example stabilizers such as lactose or sorbitol and adjuvants to enhance the immunogenic response.

Adjuvants are substances that can be used to specifically augment a specific immune response. The adjuvant and the composition are typically mixed prior to presentation to the immune system or presented separately, but into the same site of the individual being immunized. Adjuvants can be categorized into several groups based on their compositions. These groups include oil adjuvants, e.g., Freund's Complete and Incomplete adjuvants; mineral salts, for example, Al(OH)₃, AlNa(SO₄)₂, AlNH₄(SO₄), silica, kaolin, and carbon; polynucleotides such as poly Ic, poly AU acids, and CpG; and certain natural substances such as wax D from Mycobacterium tuberculosis as well as substances found in Corynebacterium parvum or Bordetella Pertussis, and member of the genus Brucella. Among those substances often used as adjuvants are the saponins, for example Quil A (Superfos A/S, Denmark), QS21 (Antigenics), and LPS derivatives such as MonoPhosphoryl Lipid A (MPL®). The formulation of vaccines are well known to those in the art. Examples of material suitable for use in vaccine compositions are provided, e.g., in Remington's Pharmaceutical Sciences, 17th Edition, A. Gennaro, Editor, Mack Publising Co., Easton, Pa., 1985).

The vaccines of the invention can be administered by a variety of routes including parenterally by injection, rapid infusion, intravenously, subcutaneously, intradermally, or intramuscularly. Administration can also be by nasopharyngeal absorption (intransopharangeally), dermoabsorption, or orally. Compositions for parenteral adminsitration include sterile adqueous or non-adqueous solutions, suspensions, and emulstions. Examples of non-aqueous solvents are propylene glycol, polyethylene glycol, vegetable oils such as olive oil, and injectable organic esters such as ethyl oleate. Carriers or occlusive dressings can be used to increase skin permeability and enhance antigen absorption. Liquid dosage forms for oral administration can generally comprise a liposome solution containing the liquid dosage form. Suitable forms for suspending liposome include emulsions, suspensions, solutions, syrups, and elixirs containing inert diluents commonly used in the art, such as purified water. Besides the inert diluents, such compositions can also include adjuvants, wetting agents, emulsifying and suspending agents, or sweetening, flavoring, or perfuming agents.

The vaccines can be administered in a number of different regimens as is apparent to one of skill in the art. The vaccines can be administered as either single or multiple dosages of an effective amount. The vaccines of the invention are administered to a patient in an amount sufficient to elicit a protective immune response and to prevent or attenuate a bacterial infection. An amount adequate to accomplish this is defined as “therapeutically effective dose.” Amounts effective for this use will depend on, e.g., the particular composition administered, the manner of administration, and factors such as the size, weight or age of the individual receiving the vaccine. Typically, effective amounts of the compositions range from 0.01-1,000 μg/ml per dose, often 0.1-500 μg/ml per dose and frequently 10-300 μg/ml perdose. For multiple administration, the timing of the dosages can vary. Typically, the dosages are administered one to two months apart.

The antibody response in an individual can be monitored by assaying for antibody titer or bactericidal activity and boosted if necessary to enhance the response. Typically, a single dose for an infant is about 10 μg of conjugate vaccine per dose or about 0.5 μg-20 μg/kilogram. Adults generally receive a dose of about 0.5 μg-20 μg/kilogram of the conjugate vaccine.

In particular applications, the vaccines can be administered maternally to confer neonatal immunity. In such an embodiment, the vaccine comprising the conjugate molecules of the invention are administered in an immunogenic amount to a female human so as to produce antibodies capable of passing into a fetus in an amount sufficient to produce protection against infection in the neonate at birth.

In another embodiment of this invention, antibodies directed against the vaccine conjugates of this invention may be used as a pharmaceutical preparation in a therapeutic or prophylactic application in order to confer passive immunity from a host individual to another individual (i.e., to augment an individual's immune response against gram-negative or gram-positive bacteria or to provide a response in immuno-compromised or immuno-depleted individuals such as AIDS patients). Passive transfer of antibodies is known in the art and may be accomplished by any of the known methods. According to one method, antibodies directed against the conjugate molecule are generated in an immunocompetent host, harvested from the host, and transfused into a recipient individual. For example, a human donor may be used to generate antibodies reactive against a conjugate of the invention. The antibodies may then be administered in therapeutically or prophylactically effective amounts to a human recipient in need of treatment, thereby conferring resistance in the recipient against bacteria which are bound by antibodies elicited by the polysaccharide component. (See, e.g, Grossman, M. and Cohen, S. N., in “Basic and Clinical Immunology”, 7th Ed., (Stites, D. P. and Terr, A. T. eds., Appleton & Lange 1991) Chapter 58)

### EXAMPLES

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

The following examples provide a description of the preparation and evaluation of a multivalent chimeric vaccine conjugate comprising Group B Streptococcus (GBS) polysaccharides joined to a Cβ carrier protein; and a Meningococcal multivalent chimeric conjugate comprising Meningococcal polysaccharides joined to a tetanus toxoid carrier.

Preparation of the Chimeric GBS Conjugate

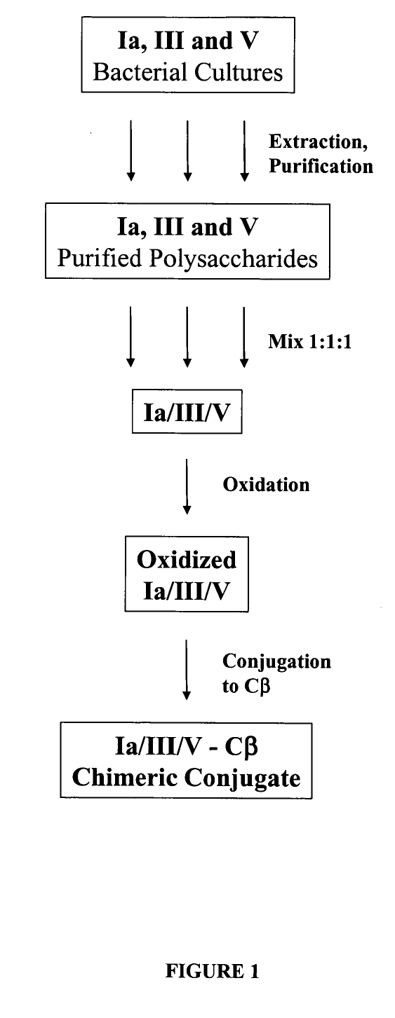

A schematic diagram showing preparation of a chimeric GBS conjugate is provided in FIG. 1 and was performed as follows:

Polysaccharide Isolation and Purification.

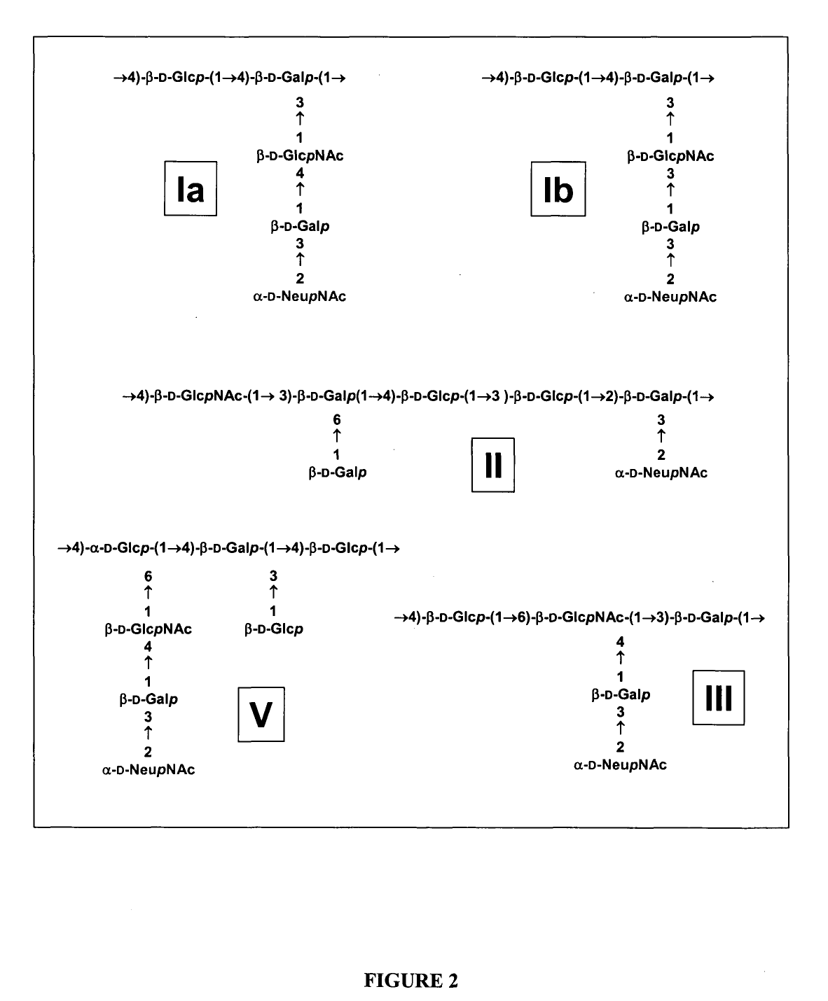

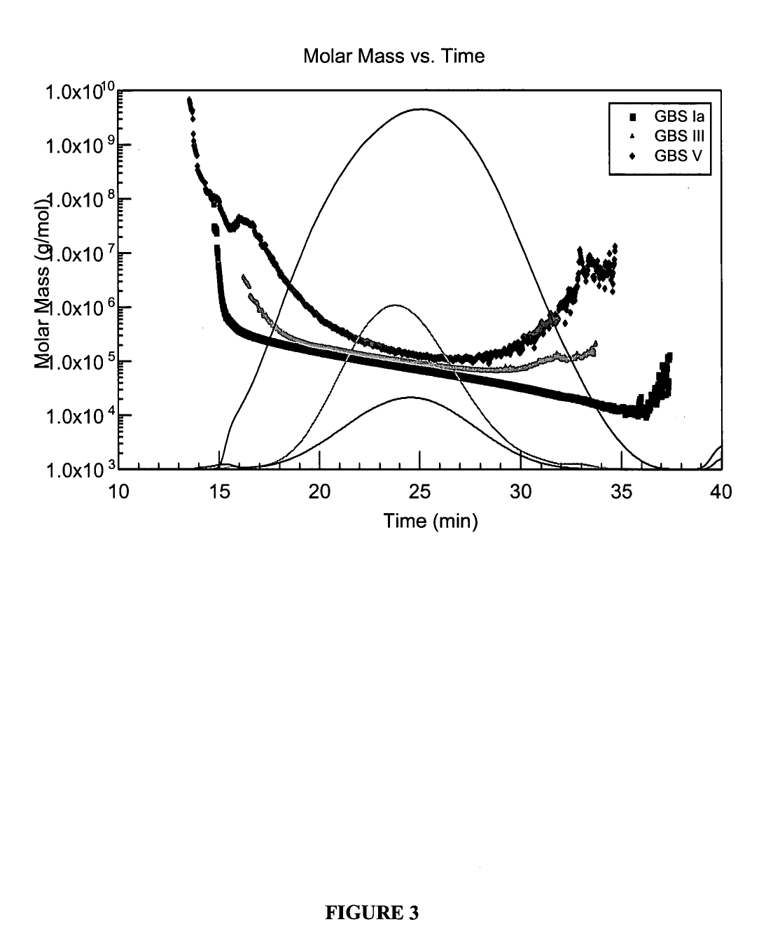

Supernatant from the culture fluid of GBS bacteria were treated with 2 N NaOH solution at 80° C. for 16 hours. The solution was then subjected to ultrafiltration using a 30,000 dalton molecular weight cutoff membrane to remove proteins and nucleic acids. The retentate was acetylated with acetic anhydride at pH8.5-10 and diafiltered. An additional treatment of the pure polysaccharide with 2N NaOH and an additional N-acetylation generated polysaccharides of the desired molecular size, i.e., from 80K-120K. GBS polysaccharides from strains Ia, Ib, II, III, and V were produced using this method. The structures of the GBS polysaccharides are shown in FIG. 2. Molar Mass determinations for GBS polysaccharides are shown in FIG. 3.

Oxidation of the Mixture of Polysaccharides.

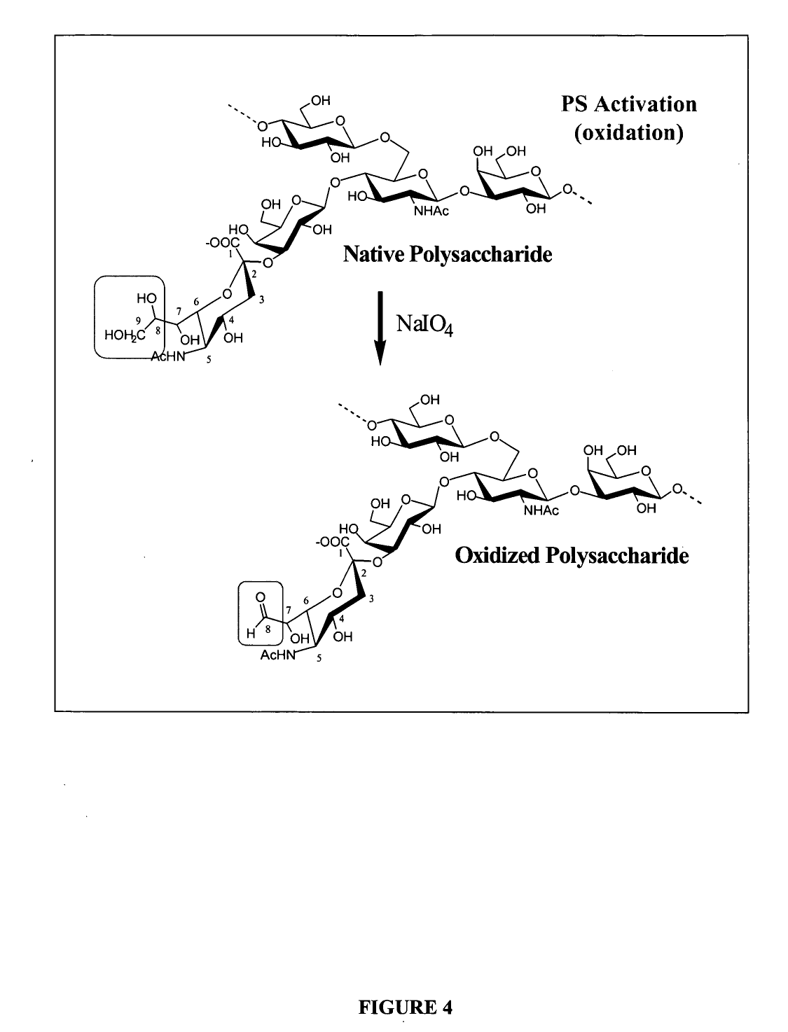

Polysaccharide (10 mg-100 mg) from GBS bacteria were mixed together in a reaction vessel at a final concentration of 10 mg/ml in 0.9% saline. The polysaccharides were oxidized with 0.5 mM-2 mM NaIO₄ (final concentration in reaction mixture) to oxidize the terminal sialic acid residue to the extent of 5%-20%. The reaction mixture was stirred at room temperature in the dark for 2 hours, capped with ethylene glycol and then diafiltered using a 10,000 dalton molecular weight cutoff membrane. The structure of an oxidized GBS polysaccharide having an aldehyde group in its terminal sialic acid is shown in FIG. 4.

Conjugation with Cβ Protein

The mixture of oxidized GBS polysaccharides in 0.25 M HEPES buffer was added to a solution of Cβ protein in the same buffer. The final concentration of the polysaccharides and protein was 12 mg/ml and 4 mg/ml, respectively. NaBH₃CN in an amount at 0.75 times the amount of polysaccharide was added to the solution. The reaction was stopped by the addition of a NaBH₄ solution. After neutralizing the excess NaBH₄, the conjugate was purified by precipitation with deoxycholate or by ultrafiltration.

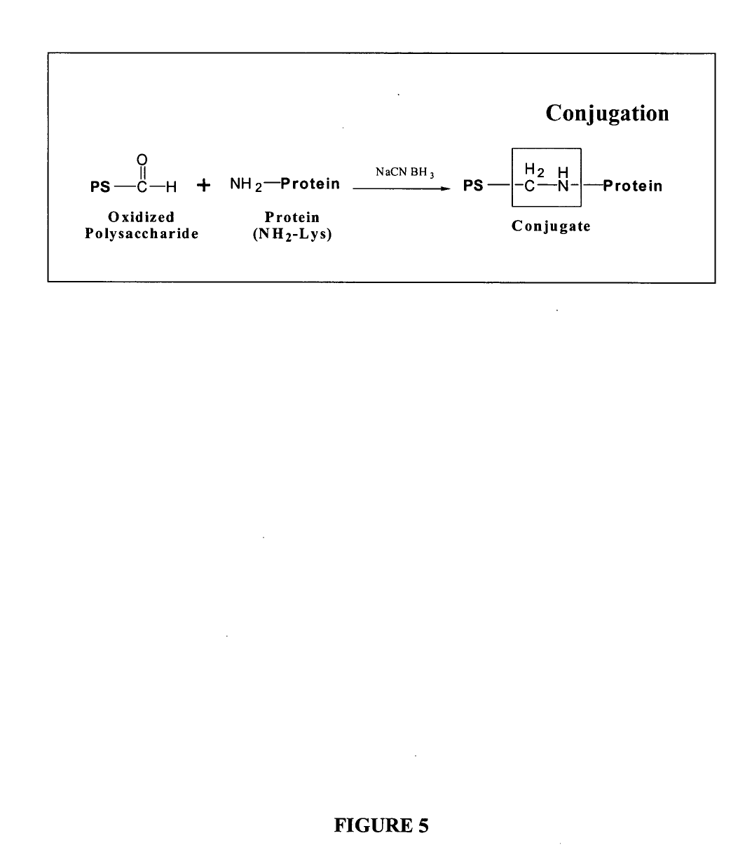

The conjugation reaction is shown in FIG. 5.

Analysis of GBS Multivalent Chimeric Conjugate Vaccine

The amount of each polysaccharide in a multivalent GBS conjugate vaccine can be quantified by using chemical derivatization and gas chromatography to distinguish particular linkages that are unique for a specific polysaccharide.

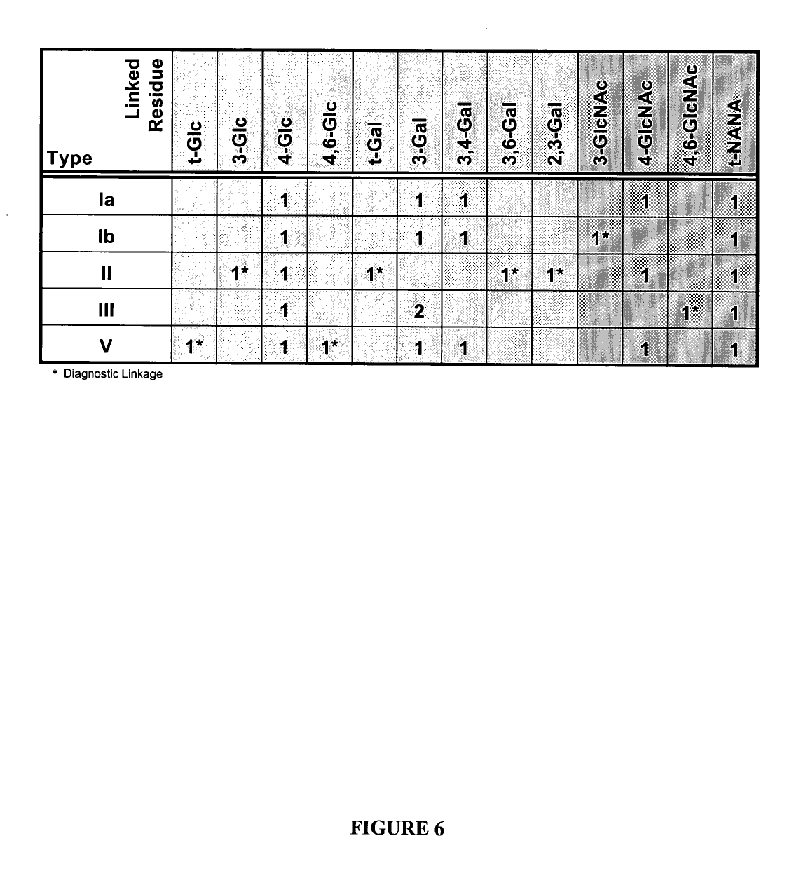

FIG. 6 shows a table of all linked monosaccharides in the types Ia, Ib, II, III, and V capsular polysaccharides. The asterisk indicates a diagnostic linkage. The neutral hexoses (Glc and Gal) can be analyzed by sequential methylation, hydrolysis, reduction, and acetylation to form partially methylated alditol acetate (PMAA-) derivatives. The amino sugar (GlcNAc) and sialic acid (NANA) can be derivatized by methylation, methanolysis, re-N-acetylation, and trimethylsilylation to form methylated trimethylsilyl (M/TMS) derivatives.

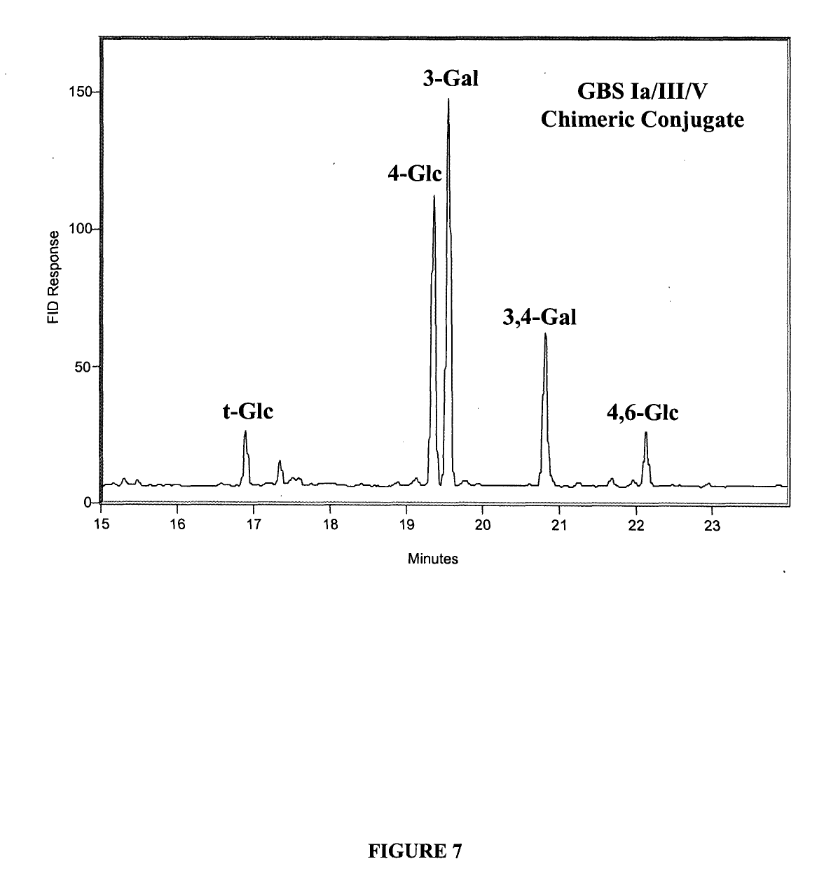

Following derivatization, the products were identified using gas chromatography. FIG. 7 shows a chromatogram of PMAA derivatives from a GBS multivalent chimeric (Ia, III, and V) conjugate. The PMAAs were chromatographed on a 30-meter RTX-1 capillary column using an HP6890 gas chromatograph with flame ionization detection. Five PMAAs, resulting from the three polysaccharides, were clearly resolved with the expected ratio of 1 (t-Glc):3(4-Glc):4(3-Gal):2(3,4-Gal): 1(4,6-Glc).

A fingerprint of PMAAs for each polysaccharide with relative integration of peak areas can be determined to quantify each polysaccharide in the multivalent conjugate. Table 1 shows the quantitative data analysis for the GBS Ia/Ill/V multivalent conjugate. In this analysis, the relative peak areas from the GBS V fingerprint in FIG. 7 are normalized relative to 4,6-Glc, a diagnostic linkage for GBS V, in the multivalent conjugate. The GBS V peak areas are then subtracted from the total peak areas of the multivalent conjugate. Similarly, the relative peak areas of GBS Ia and GBS III are sequentially subtracted, normalized against 3,4-Gal and 3-Gal, respectively. Finally, the percentage of each polysaccharide in the multivalent conjugate is calculated based on 4-Glc, and a relative value for each polysaccharide is determined.

TABLE 1

| | Peak Areas | | | | | | Relative |
|---|---|---|---|---|---|---|---|
| | t-Glc | 4-Glc | 3-Gal | 3,4-Gal | 4,6-Glc | % | Ratio |
| Total | 67.8 | 422.7 | 559.5 | 235.6 | 82.0 | | |
| GBSV | 73.1 | 123.0 | 95.1 | 95.9 | 82.0 | 27.78 | 0.83 |
| | −5.30 | 299.7 | 464.4 | 139.7 | 0.0 | | |
| GBSIa | | 143.9 | 126.1 | 139.7 | | 32.50 | 0.98 |
| | | 155.9 | 338.3 | 0.0 | | | |
| GBSIII | | 175.9 | 338.3 | | | 39.72 | 1.19 |
| | | −20.00 | 0.0 | | | | |

Ability of Multivalent Conjugate GBS Vaccine to Inhibit Binding of Individual Conjugate to Polysaccharide Antibodies

The following section describes immunological characterization of the multivalent chimeric conjugate.

In Vitro Analysis of Competitive Binding of Chimeric Conjugate vs Single Polysaccharide Conjugate Molecules

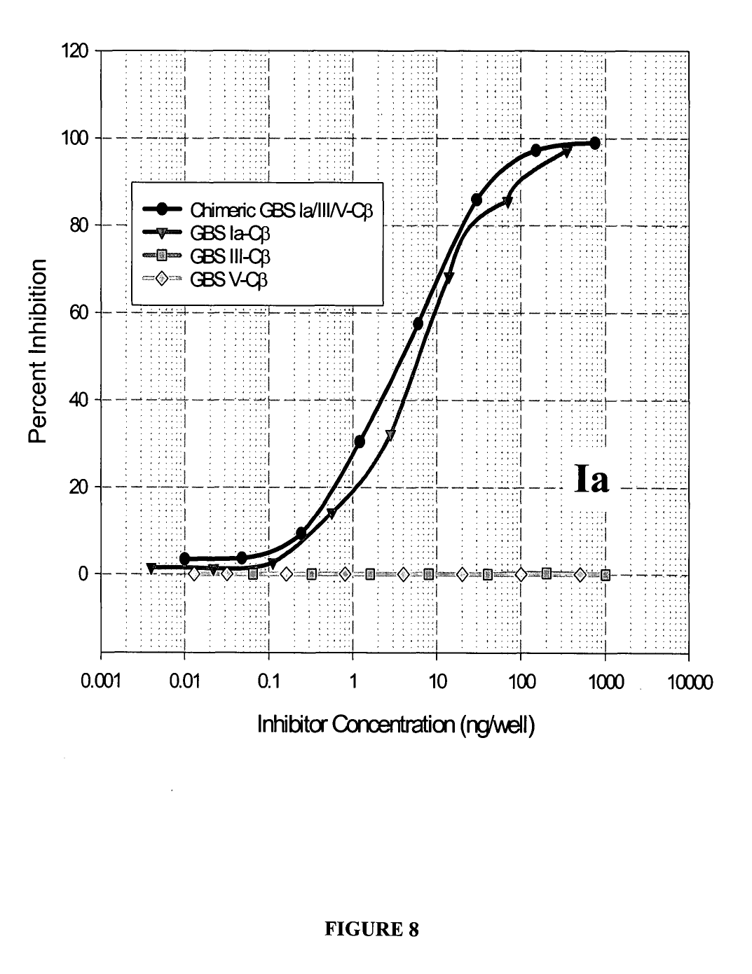

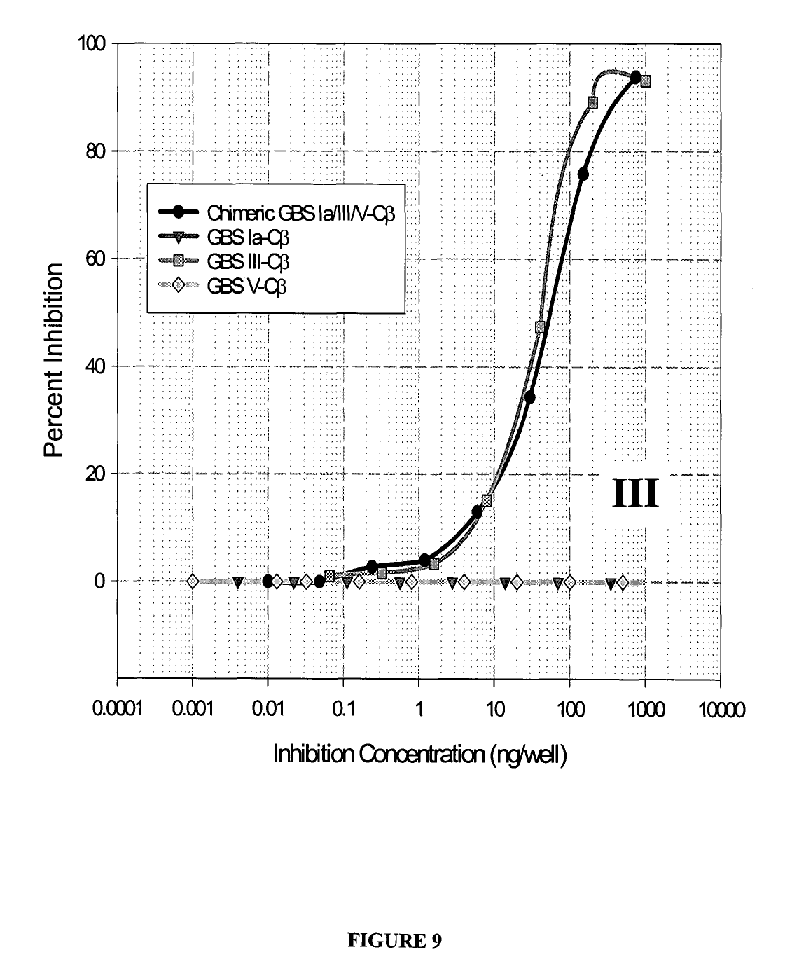

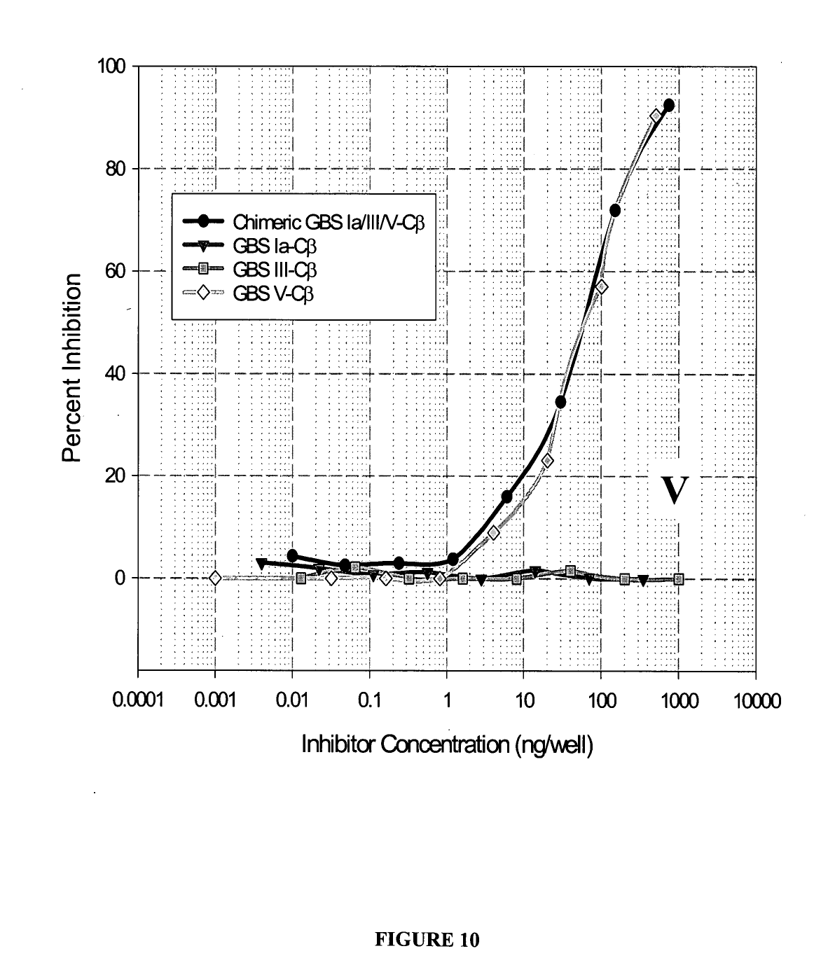

The ability of the multivalent conjugate to compete for binding was analyzed in vitro. Inhibition of binding to rabbit Anti-GBSIa antiserum was on a GBSIa-HSA-coated plate. The results showed that the multivalent conjugate and the monovalent conjugate were equally as effective in inhibiting binding of rabbit anti-gBS1 a antiserum to a GBSIA-HSA coated plate (FIG. 8). Similarly, the multivalent conjugate was equally as effective at inhibiting the binding of rabbit anti-GBSV-HSA and anti-GBSIII-HSA as their respective monovalent counterparts (FIGS. 9, 10).

Induction of a Protective Immune Response

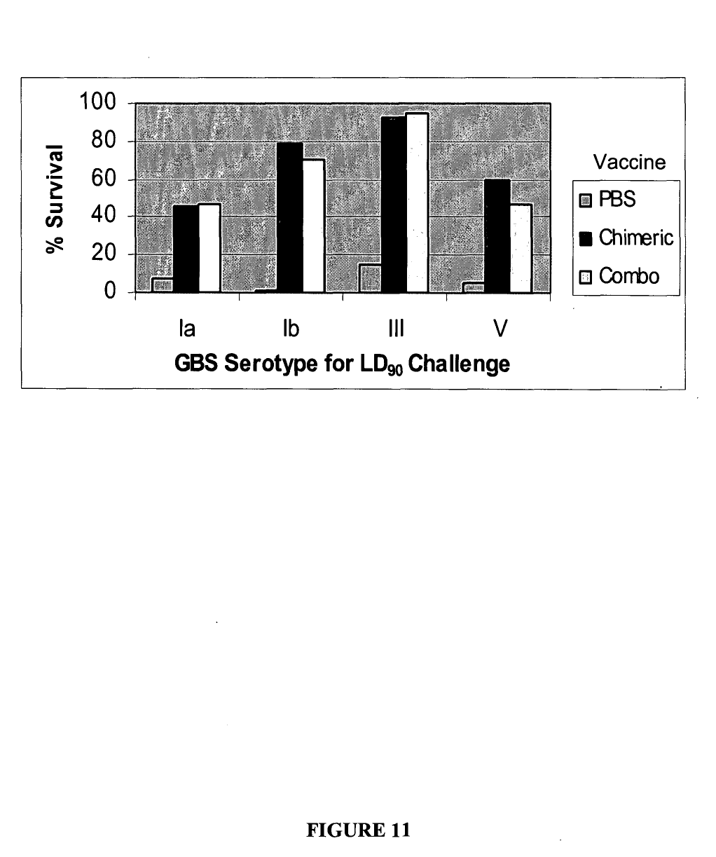

The multivalent conjugate was tested for the ability to elicit a protective immune response. The efficacy of the tetravalent chimeric conjugate prepared as described herein was evaluated in comparison to a tetravalent vaccine mixture comprising, a Ia/Ib/III/V combination vaccine, i.e., a mixture of monovalent conjugates. Animals (CD1 female mice) were inoculated with the chimeric vaccine or the combination tetravalent vaccine mix. Each animal received 1 μg of each of the conjugated type-polysaccharide, at days 0 and 21. Vaccines were adsorbed on Aluminum hydroxide (Superfos, Denmark). Mice were inpregnated at day 21. Neonates were challenged 48 hours following birth with GBS type Ia, GBS type Ib, GBS tpe III or GBS type V. The results (FIG. 11) show that the chimeric conjugate was as effective as the tetravalent vaccine mixture in eliciting a protective immune response.

Preparation and Evaluation of a Chimeric Meningococcal Multivalent Conjugate Vaccine

Meningococcal polysaccharides from serogroups C, Y, and W-135 were prepared using the methodology employed from the preparation of the GBS polysaccharides. The Meningococcal polysaccharides contain monosaccharide residues that are unique for each polysaccharide: the serogroup C polysaccharide is a homopolymer of sialic acid residues, the serogroup Y polysaccharide is made up of repeating disaccharide units of glucose and sialic acid, and the W-135 polysaccharide is made up of galactose and sialic acid repeating structures. Thus, monosaccharide composition analysis by chemical derivatization and subsequent gas chromatography (GC) can be used to differentiate and quantitate each polysaccharide in a multivalent Meninogococal conjugate vaccine.

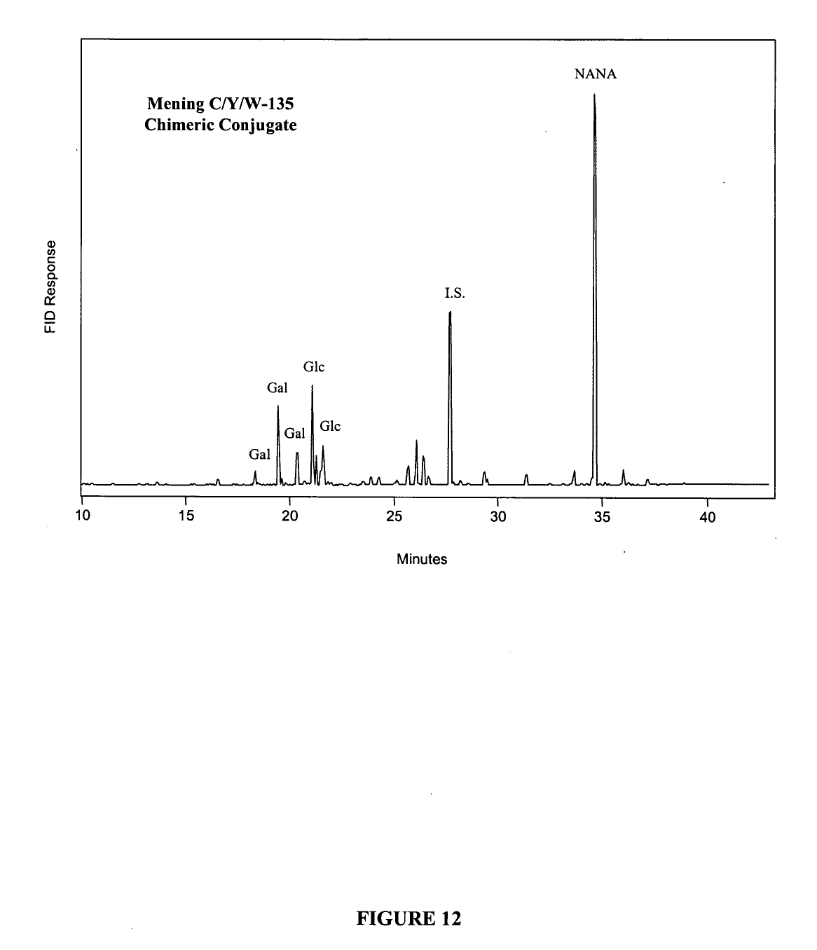

FIG. 12 shows a chromatogram of trimethylsilyl (tms) methyl glycosides from a Mening C/Y/W-135 chimeric conjugate. The sample was methanolyzed, derivatized and chromatographed on a 30-meter RTX-1 capillary column using a HP6890 gas chromatograph with flame ionization detection (GC-FID). Three monosaccharides (galactose, glucose and sialic acid), resulting from the three polysaccharides, were clearly detected.

Table 2 shows the relative polysaccharide (PS) ratios for the Mening C/Y/W-135 chimeric conjugate both prior to conjugation and after conjugate purification. Each polysaccharide in the chimeric conjugate was quantitated based on monosaccharide composition using GC-FID.

TABLE 2

| | Relative Ratio of PS | | | Relative Ratio of PS | | |
|---|---|---|---|---|---|---|
| | (starting) | | | (purified conjugate) | | |
| VACCINE | C | Y | W-135 | C | Y | W-135 |
| Mening C/Y/W-135 | 0.6 | 1.2 | 1.2 | 1.2 | 0.9 | 0.9 |
| Chimeric Conjugate | | | | | | |

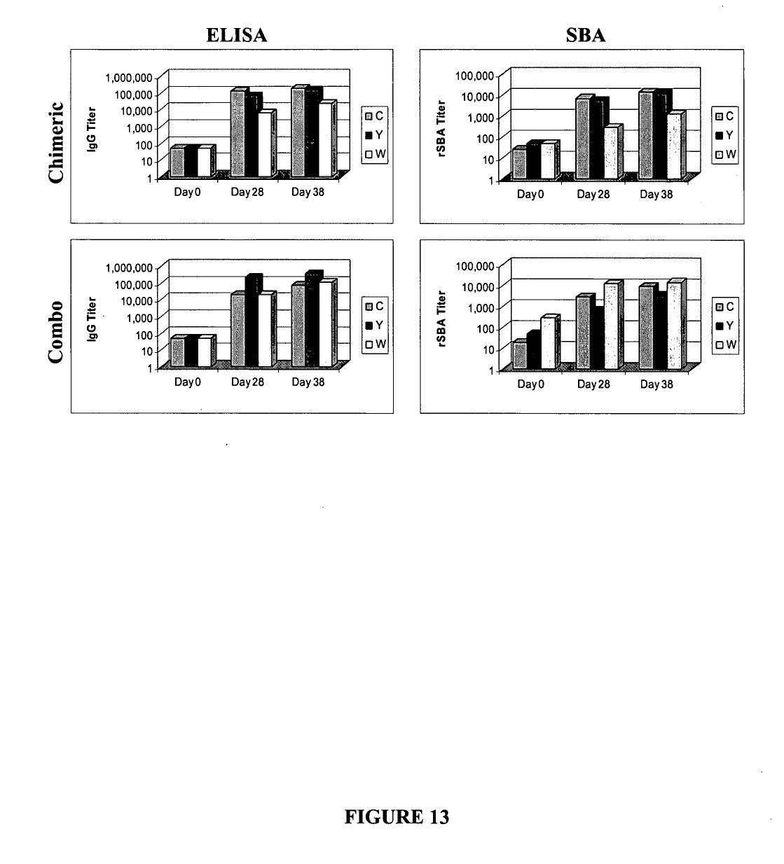

The chimeric conjugate was then compared to a combination Mening C/Y/W-135 vaccine in ELISA and serum bactericidal assays (SBA). ELISA and SBA titers generated after one injection (day 28) and after two injections (day 38) with the chimeric and combination Mening C/Y/W-135 conjugate vaccines are shown in FIG. 13. The results show that both the chimeric and combination multivalent vaccines were effective in eliciting >10-fold increases in IgG and SBA titers after 2 injections of the vaccines.

All publications and patent applications cited in this specification are herein incorporated by reference for all purposes as if each individual publication or patent application were specifically and individually indicated to be incorporated by reference.

What is claimed is:

1. A multivalent conjugate molecule comprising a carrier protein with at least three different bacterial capsular polysaccharides covalently linked to the carrier protein, wherein the molecule elicits protective antibodies.

2. The conjugate molecule of claim 1 comprising four different bacterial capsular polysaccharides covalently linked to the carrier protein.

3. The conjugate molecule of claim 1 comprising five different bacterial capsular polysaccharides covalently linked to the carrier protein.

4. The conjugate molecule of claim 1 comprising six different bacterial capsular polysaccharides covalently linked to the carrier protein.

5. The conjugate molecule of claim 1, wherein the carrier protein is selected from the group consisting of Cα, Cβ, tetanus toxoid, diphtheria toxoid, diphtheria toxoid analog CRM197, and a porin protein.

6. The conjugate molecule of claim 1, wherein the bacterial capsular polysaccharides are different Group B Streptococcus capsular polysaccharides selected from the group consisting of type Ia, type Ib, type II, type III, type V, and type VIII.

7. The conjugate molecule of claim 6, wherein the Group B Streptococcus capsular polysaccharides are type Ia, type III and type V.

8. The conjugate molecule of claim 7, wherein the carrier protein is Cβ.

9. The conjugate molecule of claim 6, wherein the bacterial capsular polysaccharides are of a size of between 80 and 120 kilodaltons.

10. The conjugate molecule of claim 6, wherein between about 5 and 20% of the sialic acid residues of the bacterial capsular polysaccharides are covalently linked to the carrier protein.

11. The conjugate molecule of claim 6, wherein the bacterial capsular polysaccharides are present in equimolar amounts.

12. The conjugate molecule of claim 1, wherein the bacterial capsular polysaccharides are Neisseria meningitidis capsular polysaccharides selected from the group consisting of A, B, C, W, and Y.

13. The conjugate molecule of claim 12, wherein the Neisseria meningitidis capsular polysaccharides are B, C, and Y.

14. The conjugate c molecule of claim 12, wherein the Neisseria meningitidis capsular polysaccharides are C, Y, and W-135.

15. The conjugate molecule of claim 12, wherein the carrier protein is a porin protein, tetanus toxoid, or CRM197.

16. The conjugate molecule of claim 14, wherein the carrier protein is tetanus toxoid.

17. A method of preparing a multivalent conjugate molecule, the method comprising covalently linking at least three different bacterial capsular polysaccharides to a carrier protein.

18. The method of claim 17, wherein covalently linking the bacterial capsular polysaccharides to the carrier protein comprises steps of:
(a) oxidizing the polysaccharides;
(b) coupling the oxidized polysaccharides to the carrier protein.

19. The method of claim 18, wherein the polysaccharides are coupled to the carrier protein by reductive animation.

20. The method of claim 18, wherein the polysaccharides are conjugated to the carrier protein by a bispacer coupling with a linker.

21. The method of claim 17, wherein the carrier protein is selected from the group consisting of Cα, Cβ, tetanus toxoid, diphtheria toxoid, diphtheria toxoid analog CRM197, and a porin protein.

22. The method of claim 17, wherein the bacterial capsular polysaccharides are different Group B Streptococcus capsular polysaccharides selected from the group consisting of type Ia, type Ib, type II, type III, type V, and type V.

23. The method of claim 22, wherein the Group B Streptococcus capsular polysaccharides are type Ia, type III, and type V.

24. The method of claim 23, wherein the carrier protein Cp.

25. The method according to claim 22, wherein between about 5 and 20% of the sialic acid residues of the bacterial capsular polysaccharides are oxidized.

26. The method according to claim 22, wherein between about 5 and 20% of the sialic acid residues of the bacterial capsular polysaccharides are coupled to protein.

27. The method of claim 17, wherein the bacterial capsular polysaccharides are Neisseria meningitidis capsular polysaccharide selected from the group consisting of A, B, C, W, and Y.

28. The method of claim 27, wherein the Neisseria meningitidis capsular polysaccharides are B, C, and Y.

29. The method of claim 27, wherein the Neisseria meningitidis capsular polysaccharides are C, Y, and W-135.

30. The method of claim 27, wherein the carrier protein is recombinant porin B, tetanus toxoid, or CRM197.

31. The method of claim 29, wherein the carrier protein is tetanus toxoid.

32. A method of preventing or attenuating an infection in a mammal, the method comprising administering to the mammal a multivalent conjugate molecule comprising a carrier protein with at least three different bacterial capsular polysaccharides covalently linked to the carrier protein, wherein the multivalent conjugate molecule is administered in an amount sufficient to elicit protective antibodies against the bacterial capsular polysaccharides.

33. The method of claim 32, wherein the carrier protein is selected from the group consisting of Cα, Cβ, tetanus toxoid, diphtheria toxoid, diphtheria toxoid analog CRM197, and a porin protein.

34. The method of claim 32, wherein the multivalent conjugate molecule is administered to prevent or attenuate an infection caused by Group B Streptococcus and the bacterial capsular polysaccharides of the conjugate molecule are different Group B Streptococcus capsular polysaccharides selected from the group consisting of type Ia, type Ib, type II, type III, type V, and type VIII.

35. The method of claim 34, wherein the Group B Streptococcus polysaccharides are type Ia, type III and type V.

36. The method of claim 35, wherein the carrier protein is Cβ.

37. The method of claim 32, wherein the multivalent conjugate molecule is administered to prevent or attenuate an infection caused by Neisseria meningitidis and the bacterial capsular polysaccharides of the conjugate molecule are different Neisseria meningitidis capsular polysaccharides selected from the group consisting of A, B, C, W, and Y.

38. The method of claim 37, wherein the Neisseria meningitidis capsular polysaccharides are B, C, and Y.

39. The method of claim 37, wherein the Neisseria meningitidis capsular polysaccharides are C, Y, and W-135.

40. The method of claim 37, wherein the carrier protein is recombinant porin B, tetanus toxoid, or CRM197.

41. The method of claim 39, wherein the carrier protein is tetanus toxoid.

42. A pharmaceutical composition comprising a multivalent conjugate molecule comprising a carrier protein with at least three different bacterial capsular polysaccharides covalently linked to the carrier protein and a pharmacological acceptable carrier, wherein the multivalent conjugate molecule is in an amount sufficient to elicit protective antibodies against the three different bacterial capsular polysaccharides.

43. The pharmaceutical composition of claim 42, wherein the carrier protein is selected from the group consisting of Cα, Cβ, tetanus toxoid, diphtheria toxoid, CRM197, and a porin protein.

44. The pharmaceutical composition of claim 42, wherein the bacterial capsular polysaccharides are different Group B Streptococcus capsular polysaccharides selected from the group consisting of type Ia, type Ib, type II, type III, type V, and type VIII.

45. The pharmaceutical composition of claim 44, wherein the Group B Streptococcus capsular polysaccharides are type Ia, type III and type V.

46. The pharmaceutical composition of claim 45, wherein the carrier protein is Cβ.

47. The pharmaceutical composition of claim 42, wherein the bacterial capsular polysaccharides of the immunogenic molecule are different Neisseria meningitidis capsular polysaccharides selected from the group consisting of A, B, C, W, and Y.

48. The pharmaceutical composition of claim 47, wherein the Neisseria meningitidis capsular polysaccharides are B, C, and Y.

49. The pharmaceutical composition of claim 47, wherein the Neisseria meningitidis capsular polysaccharides are C, Y, and W-135.

50. The pharmaceutical composition of claim 47, wherein the carrier protein is tetanus toxoid, recombinant porin B or CRM197.

51. The pharmaceutical composition of claim 49, wherein the carrier protein is tetanus toxoid.
